(19) United States
(12) Patent Application Publication
(10) Pub. No.: US 2008/0138872 A1
(43) Pub. Date: Jun. 12, 2008

(54) Processes for Producing Fermentation Products

(75) Inventors: Mads Peter Torry Smith, Raleigh, NC (US); Guillermo Coward Kelly, Raleigh, NC (US); Keith Alan McCall, Davis, CA (US)

Correspondence Address: NOVOZYMES NORTH AMERICA, INC., 500 FIFTH AVENUE, SUITE 1600, NEW YORK, NY, 10110, US

(73) Assignee: NOVOZYMES NORTH AMERICA, INC., FRANKLINTON, NC (US)

(21) Appl. No.: 11/816,722

(22) PCT Filed: Mar. 14, 2006
(86) PCT No.: PCT/US2006/009104

§ 371 Date: Aug. 21, 2007

Related U.S. Application Data
(60) Provisional application No. 60/662,639, filed on Mar. 17, 2005.
(60) Provisional application No. 60/675,244, filed on Apr. 27, 2005.

(51) Int. Cl. C12P 7/10; C12P 1/00

(52) U.S. Cl. 435/165; 435/41

(57) ABSTRACT

The present invention provides a process of producing a fermentation product comprises the steps of i) pre-treating lignocellulosic material to release or separate cellulose, hemi-cellulose and/or lignin, ii) subjecting the pre-treated material to a cellulase, iii) fermenting in the presence of a fermenting organism, wherein xylose isomerase is added in step ii) and/or step iii).

### FIELD OF THE INVENTION

The present invention relates to enzymatic processes for producing fermentation products from lignocellulosic material.

### BACKGROUND OF THE INVENTION

As the world-wide source of oil, gas, petroleum and natural gas are gradually depleting there is a desire to provide alternative energy sources.

Fuel ethanol is today produced in significant quantities by fermentation of starch-containing material. Production of ethanol from lignocellulosic material has also been suggested as such material is an inexpensive and renewable source of carbon. Lignocellulosic material (often referred to as biomass) is the major structural component of most plants and contains cellulose, hemicellulose, and lignin.

WO 2004/099381 concerns genetically modified yeast transformed with an exogenous xylose isomerase gene that enhances the yeast's ability to ferment xylose to ethanol and other desired fermentation products.

Chandrakant, P et al. Appl Microbiol Biotechnol (2000) 53:301-309 discloses simultaneous isomerization and co-fermentation of glucose and xylose by Saccharomyces cerevisiae. The yeast that ferments glucose also ferments xylulose being produced as a result of xylose isomerase action on xylose.

In order to economically exploit lignocellulosic materials it is necessary to efficiently convert xylose to ethanol or other desirable fermentation products. Therefore, there is still a need for improving processes for producing fermentation products from lignocellulosic material.

### SUMMARY OF THE INVENTION

The present invention provides processes for producing a fermentation product, especially ethanol, from lignocellulosic material.

In the first aspect, the invention relates to a process of producing a fermentation product from lignocellulosic material, wherein the process comprises the steps of:
i) pre-treating lignocellulosic material to release or separate cellulose, hemicellulose and/or lignin,
ii) subjecting the pre-treated material to a cellulase,
iii) fermenting in the presence of a fermenting organism,
   wherein xylose isomerase is added in step ii) and/or step iii).

The process of the invention may be used for producing a vast number of fermentation products including alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); furfural, gases (e.g., H₂ and CO₂), and more complex compounds, including, for example, antibiotics (e.g., penicillin and tetracycline); enzymes; vitamins (e.g., riboflavin, B₁₂, beta-carotene); hormones, and other compounds which are difficult to produce synthetically. The fermentation product may also be a consumable alcohol (e.g., beer and wine), dairy (e.g., in the production of yoghurt and cheese), leather, and tobacco industries.

### BRIEF DESCRIPTION OF THE DRAWING

FIG. 1 shows the CO₂ loss which, is proportional to the ethanol yield, of tests with and without addition of xylose isomerase to pre-treated corn stover (PCS) containing both glucose and xylose.

### DETAILED DESCRIPTION OF THE INVENTION

The present invention provides processes for producing a fermentation product from lignocellulosic material.

### Fermentation Processes of the Invention

A process of the invention generally comprises four main steps: pretreatment, hydrolysis of pretreated material, fermentation, and optionally recovery of the fermentation product in question, such as ethanol.

In the first aspect the invention relates to a process of producing a fermentation product from lignocellulosic material, wherein the process comprises the steps of:
i) pre-treating lignocellulosic material to release or separate cellulose, hemicellulose and/or lignin,
ii) subjecting the pre-treated material to a cellulase,
iii) fermenting in the presence of a fermenting organism,
   wherein xylose isomerase is added in step ii) and/or step iii).

In one embodiment the pre-treatment in step ii) is carried out using a combination of cellulase and xylose isomerase. In another embodiment, fermentation step iii) is carried out in the presence of a fermenting organism and xylose isomerase.

### Pre-Treatment—Step i)

According to the invention lignocellulosic material is pre-treated in order to improve the rate of enzyme hydrolysis and further to increase fermentation product yields. The pre-treatment in step i) is carried out to separate and/or release cellulose, hemicellulose, and lignin. The lignocellulosic material may during pre-treatment be present in an amount between 10-80 wt. %, preferably between 20-50 wt.-%. The goal is to break down the lignin seal and disrupt the crystalline structure of the lignocellulosic material. The structure of the lignocellulosic material is altered and especially polymeric constituents are made more accessible to enzyme hydrolysis in later process steps where carbohydrate polymers (i.e., cellulose and hemicellulose) are converted into fermentable hexose and pentose sugars. Pre-treatment in step i) may be carried out in any suitable way to separate and/or release cellulose, hemicellulose and/or lignin. Examples of suitable pre-treatment methods are described by Schell et al. (2003) Appl. Biochem and Biotechn. Vol. 105-108, p. 69-85, and Mosier et al. Bioresource Technology 96 (2005) 673-686, which are hereby incorporated by reference. In a preferred embodiment the lignocellulosic material is treated chemically and/or mechanically.

Chemical and/or Mechanical Treatment

In preferred embodiments of the present invention chemical treatment and mechanical treatment—the latter often referred to as physical treatment—are used alone or in combination with subsequent or simultaneous enzymatic steps to promote the separation and/or release of cellulose, hemicellulose and/or lignin from lignocellulosic material.

Preferably, the chemical and/or mechanical treatment processes are carried out, prior to the enzymatic processes, in a pre-treatment step so as to improve the enzymatic steps described herein. Alternatively, the chemical and/or mechanical treatment processes are carried out simultaneously with enzymatic step(s), such as simultaneously with addition of one or more hemicellulases to release xylose and other hemicellulose sugars. The pre-treatment step may also be carried out simultaneously with step ii) (see below) with or without addition of hemicellulase(s).

### Chemical Treatment

As used in the present invention, “chemical treatment” refers to any chemical treatment process which can be used to promote the separation and/or release of cellulose, hemicellulose and/or lignin from lignocellulosic material. Examples of suitable chemical treatment processes include, for example, acid and base treatment, dilute acid, lime and ammonia pretreatment, wet oxidation, and solvent treatment.

Preferably, the chemical treatment process is an acid treatment process, more preferably, a continuous dilute or mild acid treatment, such as, treatment with sulfuric acid, or another organic acid, such as acetic acid, citric acid, tartaric acid, succinic acid, or mixtures thereof. Other acids may also be used. Mild acid treatment means in the context of the invention that the treatment pH lies in the range from 1 to 5, preferably 1 to 3. In a specific embodiment the acid concentration is in the range from 0.1 to 2.0 wt % sulfuric acid. The acid is mixed or contacted with the lignocellulosic material and the mixture is held at a temperature in the range of 160-220° C., such as 165-195° C., for periods ranging from minutes to seconds, e.g., 1-60 minutes, such as 2-30 minutes or 3-12 minutes. Addition of sulfuric acid may be applied to remove hemicellulose. This enhances the digestibility of cellulose.

Alkaline chemical treatment with base, e.g. NaOH or Na₂CO₃, is also contemplated according to the invention.

Cellulose solvent treatment have been shown to convert 90% of cellulose to glucose and further showed that enzyme hydrolysis could be greatly enhanced when the biomass structure is disrupted. Alkaline H₂O₂, ozone, organosolv (uses Lewis acids, FeCl₃, (Al)₂SO₄ in aqueous alcohols), glycerol, dioxane, phenol, or ethylene glycol are among solvents known to disrupt cellulose structure and promote hydrolysis (Mosier et al. Bioresource Technology 96 (2005), p. 673-686).

Wet oxidation techniques involve the use of oxidizing agents, such as; sulfite based oxidizing agents and the like. Examples of solvent treatments include treatment with DMSO (Dimethyl Sulfoxide) and the like. Chemical treatment processes are generally carried out for about 5 to about 10 minutes, but may be carried out for shorter or longer periods of time.

### Mechanical Treatment

As used in the present invention, the term “mechanical treatment” refers to any mechanical or physical treatment process which can be used to promote the separation and/or release of cellulose, hemicellulose and/or lignin from lignocellulosic material. Mechanical treatment includes comminution (mechanical reduction in biomass particulate size, steam explosion and hydrothermolysis. Comminution includes dry and wet and vibratory ball milling. Preferably, a mechanical treatment process involves a process which uses high pressure and/or high temperature (steam explosion). In context of the invention high pressure means pressure in the range from 300 to 600, preferably 400 to 500, such as around 450 psi. In context the invention high temperature means temperatures in the range from about 100 to 300° C., preferably from about 140 to 235° C. In a specific embodiment impregnation is carried out at a pressure of about 450 psi and at a temperature of about 235° C. More preferably, the mechanical process is a batch-process, steam gun hydrolyzer system which uses high pressure and high temperature, such as, using the Sunds Hydrolyzer (available from Sunds Defibrator AB (Sweden).

### Combined Chemical and Mechanical Treatment

In preferred embodiments, both chemical and mechanical treatments are carried out involving, for example, both dilute or mild acid treatment and high temperature and pressure treatment. The chemical and mechanical treatments may be carried out sequentially or simultaneously, as desired.

Accordingly, in a preferred embodiment, the process comprises the step of pre-treating lignocellulosic material using both chemical and mechanical treatment to promote the separation and/or release of cellulose, hemicellulose and/or lignin.

In a preferred embodiment the pretreatment step i) is carried out as a dilute or mild acid steam explosion step. In another preferred embodiment the pretreatment step i) is carried out as an ammonia fiber explosion step (or AFEX pre-treatment step).

In an embodiment of the invention the fermentability of, e.g., dilute-acid hydrolyzed, lignocellulosic material, such as corn stover, is improved by steam stripping in order to detoxify the material.

### Hydrolysis—Step ii)

As mentioned above lignocellulosic material is pre-treated to separated and/or released cellulose, hemicellulose and/or lignin. In step ii) the carbohydrate polymers are converted into monomeric sugars.

Cellulose can be hydrolyzed enzymatically using a cellulase (see “Cellulase”-section below) or chemically (see the “Chemical treatment”-section above) to glucose.

Hemicellulose polymers can be broken down by hemicellulases or acid hydrolysis to release its five and six carbon sugar components. The six carbon sugars (hexoses), such as glucose, galactose and mannose, can readily be fermented to, e.g., ethanol, acetone, butanol, glycerol, citric acid and fumaric acid, by a suitable fermenting organisms including yeast. Preferred for ethanol fermentation is yeast of the species Saccharomyces cerevisiae, which is resistant towards high levels of ethanol, i.e., up to, e.g., about 10-15 vol. % or more ethanol.

However, five carbon sugars (pentoses), such as xylose, which generally is comprised in significant amounts in lignocellulosic material, such as hardwood, agricultural residues, and grasses, can only be fermented to, e.g., ethanol, by few fermenting organisms and at low yields.

In one embodiment of the invention the pre-treated lignocellulosic material is present in amounts of around 10-50 wt-%, preferably around 15-35 wt.-%, especially around 20-30 wt-%, in step ii).

In one embodiment pre-treatment step ii) may be carried out in the presence of cellulase or a combination of cellulase and xylose isomerase. Xylose isomerase may also be present during the following fermentation step iii). In a preferred embodiment the pre-treated lignocellulosic material obtained in step i) is initially treated with a hemicellulase, preferably a xylanase, esterase, cellobiase, or combination thereof. Alternatively, step ii) is carried out in the presence of a combination of hemicellulase and/or cellulase and/or xylose isomerase. Hemicellulase may be added to provide more available xylose and other sugars, including glucose, from the hemicellulose fraction. However, hemicellulase treatment is not mandatory according to the invention.

Cellulase hydrolyses cellulose into glucose. The xylose isomerase converts xylose into xylulose, which can be converted to the desired fermentation product, such as ethanol, during fermentation by yeasts, such as Saccharomyces cerevisiae. It is believed that adding xylose isomerase in step ii) and/or iii) results in reduced xylose inhibition of cellulase action. In other words, by converting xylose into xylulose, inhibition of the cellulase is reduced.

In a preferred embodiment xylose isomerase is added before cellulase. In a preferred embodiment xylose is continuously converted into xylulose and then fermented. By reducing the xylose content through isomerization into xylulose the cellulose conversion rate can be increased. This reduces the process time for producing the desired fermentation product, such as ethanol. Further, the lignocellulosic raw material is utilized more efficiently, since lignocellulosic material, such as corn stover, contains approximately about 35 wt-% cellulose and 25% xylan.

The enzymatic treatment is carried out in a suitable aqueous environment under conditions which can readily be determined by one skilled in the art. In a preferred embodiment step ii) is carried out at optimal conditions for the cellulase and/or xylose isomerase in question.

Suitable process time, temperature and pH conditions can readily be determined by one skilled in the art present invention. Preferably, step ii) is carried out at a temperature between 30 and 70° C., preferably between 40 and 60° C., especially around 50° C. Preferably, step ii) is carried out at a pH in the range from 3-8, preferably pH 4-6, especially around pH 5. Preferably, step ii) is carried out for between 8 and 72 hours, preferably between 12 and 48 hours, especially around 24 hours.

Fermentation—Step iii)

Step iii) is a fermentation step and includes, without limitation, fermentation methods or processes used to produce any fermentation product, including alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. Step iii) may also be a fermentation step used in the consumable alcohol industry (e.g., beer and wine), dairy industry (e.g., fermented dairy products), leather industry and tobacco industry. In a preferred embodiment the fermentation step iii) is an alcohol fermentation processes. Preferred the fermentation step iii) is anaerobic.

In an embodiment one or more of the enzymes, i.e., hemicellulase, cellulase, xylose isomerase, added during step ii) will also be active during fermentation. However, it is also contemplated to add more hemicellulase, cellulase, xylose isomerase, or a combination thereof, during fermentation step iii). In other words, step iii) may in one embodiment be carried out as a simultaneous isomerization and fermentation step, so that the xylose isomerase converts xylose to xylulose and the fermenting organism, such as yeast, ferments xylulose to the desired fermentation product, such as ethanol.

### Fermenting Organism

The term “fermenting organism” refers to any organism, including bacterial and fungal organisms, suitable for producing a desired fermentation product. Especially suitable fermenting organisms according to the invention are able to ferment, i.e., convert, sugars, such as xylulose and/or glucose, directly or indirectly into the desired fermentation product. Examples of fermenting organisms include fungal organisms, such as yeast. Preferred yeast includes strains of Saccharomyces spp., in particular a strain of Saccharomyces cerevisiae or Saccharomyces uvarum; a strain of Pichia, preferably Pichia stipitis, such as Pichia stipitis CBS 5773; a strain of Candida, in particular a strain of Candida utilis, Candida diddensii, or Candida boidinii, which are capable of fermenting both glucose and xylulose into ethanol. Other contemplated yeast includes strains of Zymomonas; Hansenula, in particular H. anomala; Klyveromyces, in particular K. fragilis; and Schizosaccharomyces, in particular S. pombe.

Commercially available yeast include, e.g., RED STAR®/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties).

### Simultaneous Hydrolysis and Fermentation

In an embodiment the xylose isomerase used in a process of the invention has significant activity around temperatures suitable for the fermenting organism. In such case the hydrolysis and fermentation in steps ii) and iii) may be carried out simultaneously.

A “significant activity” means at least 50% of the activity obtained at optimal fermentation conditions, preferably at least 60% activity, more preferably at least 70% activity, more preferably at least 80% activity, even more preferably at least 90% activity, even more preferably at least 95% of the activity at optimal fermentation conditions. Optimal fermentation conditions is in a preferred embodiment a temperature from 28 and 40° C., preferably around 32° C., and at a pH from 3 to 7, preferably from around 3.5 to around 5.

In general, if the xylose isomerase requires conditions significantly different from what is optimal for the fermenting organism the hydrolysis step is finalized before fermentation is initiated.

In cases where the xylose isomerase is derived from Candida boidinii, preferably Candida boidinii Kloeckera, especially Candida boidinii (Kloeckera 2201) (DSM70034 or ATCC48180) (mentioned below) simultaneous hydrolysis and fermentation process may be carried out from around 28 to around 40° C., preferably from around 30 to around 38° C., especially around 32° C., and at a pH from around 3 to around 7, preferably from around 3.5 to around 5.

### Lignocellulosic Material

Lignocellulosic materials are heterogeneous complexes of carbohydrate polymers (cellulose and hemicellulose) and lignin.

Cellulose, like starch, is a homogenous polymer of glucose. However, unlike starch, the specific structure of cellulose favors the ordering of the polymer chains into tightly packed, highly crystalline structures, that are water insoluble and resistant to depolymerization. Hemicellulose is, dependent on the species, a branched polymer of glucose or xylose, substituted with arabinose, xylose, galactose, furose, mannose, glucose or glucuronic acid (Mosier et al. Bioresource Technology 96 (2005) 673-686). Lignin is an insoluble high molecular weight material of aromatic alcohols that strengthens the lignocellulosic material. In general lignin contains three aromatic alcohols (coniferyl alcohol, sinapyl and p-coumaryl). In additions, grass and dicot lignin also contain large amounts of phenolic acids such as p-coumaric and ferulic acid, which are esterified to alcohol groups of each other and to other alcohols such as sinapyl and p-coumaryl alcohols. Lignin is further linked to both hemicelluloses and cellulose forming a physical seal around the latter two components that is an impenetrable barrier preventing penetration of solutions and enzymes (Howard R. L et al. (African Journal of Biotechnology Vol. 2 (12) pp. 602-619, December 2003).

Any suitable lignocellulosic material may be used according to the present invention. Examples of contemplated lignocellulosic materials suitable for use in a process of the invention, include stover, cobs, stalks, husks, bran, seeds, peels, fruit stones, shells, bagasse, manure, wood residues, barks, leaves, wood chips, wood shavings, saw dust, fiber waste, newspapers, office paper, cardboard, grass etc. In a preferred embodiment of the invention the lignocellulosic material comprise corn stover, corn fiber, pine wood, wood chips, popular, wheat straw, switch grass, and paper, or mixtures thereof.

### Enzymes

### Hemicellulase

In an embodiment of the invention the pre-treated lignocellulosic material is treated with a hemicellulase. Any hemicellulase suitable for use in hydrolyzing hemicellulose into xylose may be used. Preferred hemicellulases for use in a process of the present invention include xylanases, arabinofuranosidases, acetyl xylan esterase, feruloyl esterase, glucuronidases, endo-galactanase, mannases, endo or exo arabinases, exo-galactanses, and mixtures thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes A/S, Denmark). The hemicellulase is added in an amount effective to hydrolyze hemicellulose into xylose, such as, in amounts from about 0.001 to 0.5 wt-% of total solids (TS), more preferably from about 0.05 to 0.5 wt-% of TS.

### Cellulase

Any cellulase that is capable of hydrolyzing cellulose into glucose may be used according to the present invention. The cellulase activity used according to the invention may be derived from any suitable origin; preferably, the cellulase is of microbial origin, such as derivable from a strain of a filamentous fungus (e.g., Aspergillus, Trichoderma, Humicola, Fusarium, Thielavia). Preferably, the cellulase composition acts on both cellulosic and lignocellulosic material. Preferred cellulases for use in the present invention include exo-acting cellulases and cellobiases, and combinations thereof. More preferably, the treatment involves the combination of an exo-acting cellulase and a cellobiase. Preferably, the cellulases have the ability to hydrolyze cellulose or lignocellulose under acidic conditions of below pH 7. Examples of cellulases suitable for use in the present invention include, for example, CELLULCLAST™ (available from Novozymes A/S), NOVOZYM™ 188 (available from Novozymes A/S). Other commercially available preparations comprising cellulase which may be used include CELLUZYME™, CEREFLO™ and ULTRAFLO™ (Novozymes A/S), LAMINEX™ and SPEZYME™ CP (Genencor Int.) and ROHAMENT™ 7069 W (from Röhm GmbH).

The cellulase enzyme(s) is(are) added in step ii) in amounts effective to hydrolyze cellulose from pretreated lignocellulosic material into glucose, such as, to provide an activity level in the range from 0.1-100 FPU per gram total solids (TS), preferably 0.5-50 FPU per gram TS, especially 1-20 FPU per gram TS or in an amount of 0.1-100 mg enzyme protein per gram total solids (TS), preferably 0.5-50 mg enzyme protein per gram TS, especially 1-20 mg enzyme protein per gram TS.

### Xylose Isomerase

Xylose isomerases (D-xylose ketoisomerase) (E.C. 5.3.1.5.) are enzymes that catalyze the reversible isomerization reaction of D-xylose to D-xylulose. Some xylose isomerases also convert the reversible isomerization of D-glucose to D-fructose. Therefore, xylose isomarase is sometimes referred to as “glucose isomerase”.

A xylose isomerase used in a process of the invention may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoplanes, Bacillus and Flavobacterium, and Thermotoga, including T. neapolitana (Vieille et al. Appl. Environ. Microbiol. 1995, 61 (5), 1867-1875) and T. maritima.

Examples of fungal xylose isomerases are derived species of Basidiomycetes.

A preferred xylose isomerase is derived from a strain of yeast genus Candida, preferably a strain of Candida boidinii, especially the Candida boidinii xylose isomerase disclosed by, e.g., Vongsuvanlert et al., (1988), Agric. Biol. Chem., 52(7): 1817-1824. The xylose isomerase may preferably be derived from a strain of Candida boidinii (Kloeckera 2201), deposited as DSM 70034 and ATCC 48180, disclosed in Ogata et al. Agric. Biol. Chem., Vol. 33, p. 1519-1520 or Vongsuvanlert et al. (1988) Agric. Biol. Chem., 52(2), p. 1519-1520.

In one embodiment the xylose isomerase is derived from a strain of Streptomyces, e.g., derived from a strain of Streptomyces murinus (U.S. Pat. No. 4,687,742); S. flavovirens, S. albus, S. achromogenus, S. echinatus, S. wedmorensis all disclosed in U.S. Pat. No. 3,616,221. Other xylose isomerases are disclosed in U.S. Pat. No. 3,622,463, U.S. Pat. No. 4,351,903, U.S. Pat. No. 4,137,126, U.S. Pat. No. 3,625,828, HU patent no. 12,415, DE patent 2,417,642, JP patent no. 69,28,473, and WO 2004/044129 (which as all incorporated by reference.

The xylose isomerase may be either in immobilized or liquid form. Liquid form is preferred.

The xylose isomerase is added to provide an activity level in the range from 0.01-100 IGIU per gram total solids.

Examples of commercially available xylose isomerases include SWEETZYME™ T from Novozymes A/S, Denmark.

### Recovery

In a preferred embodiment the fermentation product is recovered, e.g., by distilled using any method know in the art. The fermentation mash may be distilled to extract the fermentation product, in particular ethanol. The end product obtained may according to the invention be used as, e.g., fuel ethanol; drinking ethanol, i.e., potable neutral spirits; or industrial ethanol.

Further details on how to carry out milling, liquefaction, saccharification, fermentation, distillation, and ethanol recovery are well known to the skilled person.

Various modifications of the invention described herein will become apparent to those skilled in the art. Such modifications are intended to fall within the scope of the appending claims.

### Materials and Methods

Xylose isomerase: Immobilized xylose isomerase derived from Streptomyces murinus and disclosed in U.S. Pat. No. 4,687,742.
Xylose isomerase: derived from Candida boidinii (Kloeckera 2201 aka DSM 70034 aka ATCC 48180) described in Vongsuvanlert et al (1988) Agric. Biol. Chem. 52(2), p. 419-426,
Cellulase: Cellulase complex derived from Trichoderma reeseii and is commercially available from Novozymes A/S, Denmark, as CELLUCLAST™ 1.5 L
Cellobiase: Cellobiase derived from Aspergillus niger and available from as NOVOZYM™ 188 from Novozymes A/S, Denmark.

### Yeast:

Red Star™ available from Red Star/Lesaffre, USA

### Methods:

### Xylose/Glucose Isomerase Assay (IGIU)

1 IGIU is the amount of enzyme which converts glucose to fructose at an initial rate of 1 micromole per minute at standard analytical conditions.

Standard Conditions:

| | |
|---|---|
| Glucose concentration: | 45% w/w |
| pH: | 7.5 |
| Temperature: | 60° C. |
| Mg2+ concentration: | 99 mg/l (1.0 g/l MgSO4*7H2O) |
| Ca2+ concentration | <2 ppm |
| Activator, SO2 concentration: | 100 ppm (0.18 g/l Na2S2O5) |
| Buffer, Na2CO3, concentration: | 2 mM Na2CO3 |

### Measurement of Cellulase Activity Using Filter Parer Assay (FPU Assay)

### 1. Source of Method

1.1 The method is disclosed in a document entitled “Measurement of Cellulase Activities” by Adney, B. and Baker, J. 1996. Laboratory Analytical Procedure, LAP-006, National Renewable Energy Laboratory (NREL). It is based on the IUPAC method for measuring cellulase activity (Ghose, T. K., Measurement of Cellulse Activities, Pure & Appl. Chem. 59, pp. 257-268, 1987.

### 2. Procedure

2.1 The method is carried out as described by Adney and Baker, 1996, supra, except for the use of a 96 well plates to read the absorbance values after color development, as described below.

### 2.2 Enzyme Assay Tubes:

2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.

### 2.3 Blank and Controls

2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.

### 2.4 Glucose Standards

2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows:
   G1=1.0 mL stock+0.5 mL buffer=6.7 mg/mL=3.3 mg/0.5 mL
   G2=0.75 mL stock+0.75 mL buffer=5.0 mg/mL=2.5 mg/0.5 mL
   G3=0.5 mL stock+1.0 mL buffer=3.3 mg/mL=1.7 mg/0.5 mL
   G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.

### 2.5 Color Development

2.5.1 Following the 60 min. incubation and addition of DNS, the tubes are all boiled together for 5 mins. in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH2O in a 96-well plate. Each well is mixed, and the absorbance is read at 540 nm.
   2.6 Calculations (examples are given in the NREL document)
2.6.1 A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.
2.6.2 A plot of glucose produced (mg/0.5 mL) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows:

FPU/mL=0.37/enzyme dilution producing 2.0 mg glucose

### Determination of Cellobiase Activity (CBU)

Cellobiase (beta-glucosidase EC 3.2.1.21) hydrolyzes beta-1,4 bonds in cellobiose to release two glucose molecules. The amount of glucose released is determined specifically and quantitatively using the hexokinase method as follows:

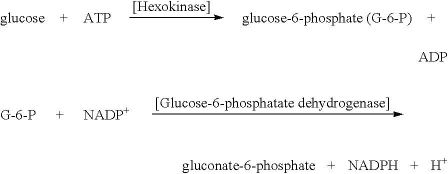

The increase in absorbance is then measured at 340 nm as the absorbance value for NADPH is high at this wavelength.

Reaction conditions
Reaction:

| Temperature | 40° C. |
|---|---|
| pH | 5.0 |

Detection:

| | |
|---|---|
| Reaction time | 15 minutes |
| Wavelength | 340 nm |

One cellobiase unit (CBU) is the amount of enzyme, which releases 2 micro mole glucose per minute under the standard conditions above with cellobiose as substrate.

A folder (EB-SM-0175.02/02) describing this analytical method in more detail is available on request from Novozymes A/S, Denmark, which folder is hereby included by reference.

Various references are cited herein, the disclosures of which are incorporated by reference in their entireties.

Purification of Xylose Isomerase from Canida boidinii

Purification of Candida boidinii is described in Vongsuvanlert et al., (1998), Agric. Biol. Chem. 52(7), p. 1817-1824. Description of the cell culture can be found in Vongsuvanlert et al. (1988) Agric. Biol. Chem. 52(2), p. 419-426.

| Basal medium, recipe for 100 ml: | for 4 L |
|---|---|
| 0.4 g of NH₄Cl | 16.0 g |
| 0.1 g of KH₂PO₄ | 4 g |
| 0.1 g of K₂HPO₄, | 4 g |
| 0.05 g of MgSO₄—7H₂O | 2 g |
| 0.2 g of yeast extract, and | 8 g |
| 0.3 g of Polypepton (Daigo) | 12 g |
| For inoc. (1 g of D-glucose) | For inoc. (40 g) |
| For growth (2.0 g of D-xylose) | For growth (80 g) |
| add ddH₂O to bring volume to 100 ml | to 4 L |
| then pH to pH 5.5 | |

### Inoculum

The inoculum is prepared by growing Candida boidinii (Kloeckera 2201 aka DSM 70034 aka ATCC 48180) cells in 100 ml of the basal medium containing 1% w/w of D-glucose in a 500 ml baffled flask for 24 hours at 28° C. under shaking at 200 rpm.

### Cultivation

The inoculum culture is added at a dilution of 5 ml inoculum culture per 500 ml growth media to a growth media consisting of 500 ml of the basal medium containing 2% (w/v) D-xylose in a 2 L baffled shaker flask. Cultivation is done at 28° C. under reciprocal shaking at 200 rpm, for 45 hours.

### Preparation of Cell-Free Extract:

Cells are collected by centrifugation and washed twice with 50 mM KHPO₄, pH 7.0, with 0.25 mM DTT. The cell paste is then suspended in the same buffer at the dilution of 1 mL buffer per gram of cell paste. The mixture is loaded into an ice-chilled BioSpec BeadBeater chamber, to which 0.52 mm glass beads are added at the ratio of 4 grams beads per gram of cell paste. A small amount of protease inhibitors is added and then the cell-buffer-bead mixture is beat in the BioSpec BeadBeater for 4 cycles of 1 minute beating then 1 minute resting on ice. Separation of beads, cell pellet, and supernatant is performed by centrifugation at 4° C. After centrifugation, the resultant supernatant solution was used as the cell-free extract.

### Purification of Xylose Isomerase:

All purification steps are to be performed at 4° C. and with centrifugation at 20,000×g for 20 minutes. The buffer is 50 mM KHPO₄, pH 7.0, containing 0.25 mM DTT, unless otherwise stated. Any concentration of the enzyme is by Amicon ultrafiltration with a YM-30 membrane.

Step 1: Protamine sulfate treatment. A one-fifth volume of a 2% protamine sulfate solution was added drop-wise to the cell-free extract, the pH being adjusted to 7.0 with 10% NH4OH under stirring, followed by standing for 30 min. The precipitate formed was removed by centrifugation.

Step 2. Ammonium sulfate saturation to 30%. To the resultant supernatant, solid ammonium sulfate is added to 30% saturation (176 g/L) with stirring, the pH being adjusted to 7.0 with at 10% NH4OH solution. After standing for 1 hr, the precipitate formed is removed by centrifugation and the supernatant is used in the next step.

Step 3. Ammonium sulfate saturation to 80%. To the resultant supernatant, solid ammonium sulfate is added to 80% saturation with stirring, the pH being adjusted to 7.0 with at 10% NH₄OH solution. After standing over-night, the precipitate formed was collected by centrifugation and then dissolved in a minimum volume of buffer. The supernatant is not used in any following steps. It is the resuspended pellet that is the subject of further purification. The resuspended pellet solution is dialyzed against is 50 mM KHPO₄, pH 7.0, containing 0.25 mM DTT over-night.

Step 4. MnCl₂ treatment. The dialyzed protein solution is centrifuged and then 1 M MnCl₂-4H₂O was added to the concentration of 5% (w/v), with the pH being adjusted to 7.0 with 10% NH₄OH under stirring, followed by standing for 30 minutes. The precipitate formed was removed by centrifugation and the resultant supernatant was concentrated.

The protein solution now contains xylose isomerase of sufficient purity for initial activity assays. Further purification of the sample can be carried out by standard column chromatography techniques.

### EXAMPLES

### Example 1

### Xylose Fermentation of Corn Stover

The impact of xylose isomerase on Pretreated Corn Stover (PCS) fermentation containing both glucose and xylose was investigated.

Corn Stover was first pretreated with about 0.5% dilute sulfuric acid and then subjected to steam explosion. The pre-treated material was not pressed to remove hydrolysates and therefore contained all solubles from pretreatment.

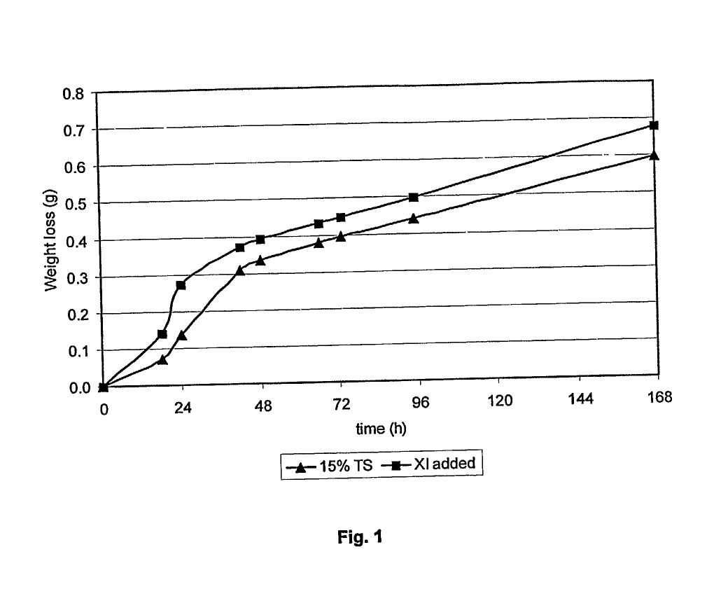

15 wt.-% TS PCS was hydrolyzed at 50° C. in the presence of Trichoderma reeseii cellulase (5 FPU/g TS) supplemented with Aspergillus niger cellobiase (1.5 CBU per FPU) and about 13 IGIU per gram TS immobilized xylose isomerase derived from Streptomyces murinus at pH 5. Fermentation at 32° C. was started after 48 hours of hydrolysis by inoculating with yeast (Saccharomyces cerevisiae—RED STAR™) at 10% pitch, i.e., ratio of propagate to total volume, to secure a high initial cell count. A growth media containing 1% yeast extract and 1% peptone was used as a nutrient and nitrogen source. The CO₂ loss was determined which is proportional to the ethanol production. The experiment was also carried out without addition of xylose isomerase. The result of the tests is displayed in FIG. 1.

### Example 2

### Simultaneous Xylose Isomeration and Fermentation

Corn Stover is first pretreated with about 0.5% dilute sulfuric acid and then subjected to steam explosion. The pre-treated material is not pressed or washed to remove liquid hydrolysates and therefore contained all solubles from pretreatment.

Subsequently, 15 wt.-% TS PCS is hydrolyzed at 50° C. in the presence of Trichoderma reeseii cellulase (5 FPU/g TS) supplemented with Aspergillus niger cellobiase (1.5 CBU per FPU) at pH 5. Finally, fermentation is carried out in the presence of about 13 IGIU per gram TS xylose isomerase derived from Candida boidinii (Kloeckera no. 2201) at pH 5. Fermentation at 32° C. is initiated after 48 hours of hydrolysis by inoculating with yeast (Saccharomyces cerevisiae—RED STAR™) as the fermenting organism at 10% pitch, i.e., ratio of propagate to total volume, to secure a high initial cell count. A growth media containing 1% yeast extract and 1% peptone is used as a nutrient and nitrogen source. Fermentations are monitored measuring xylose, xylulose, glucose and ethanol using HPLC-RI. Controls are included where xylose isomerase is not added to the fermentation to determine the production of ethanol when xylose is not utilized.

### Example 3

### Simultaneous Hydrolysis, Xylose Isomeration and Fermentation

Corn Stover is first pretreated with about 0.5% dilute sulfuric acid and then subjected to steam explosion. The pre-treated material is not pressed or washed to remove liquid hydrolysates and therefore contained all solubles from pretreatment.

15 wt.-% TS PCS is converted in a Simultaneous Saccharification and Fermentation (SSF) setup using a Trichoderma reeseii cellulase (5 FPU/g TS) supplemented with Aspergillus niger cellobiase (1.5 CBU per FPU) and about 13 IGIU per gram TS xylose isomerase derived from Candida boidinii (Kloeckera no. 2201). The simultaneous enzyme treatment and fermentation is carried out at 32° C. and pH 5 using yeast (Saccharomyces cerevisiae—RED STAR™) as the fermenting organism at 10% pitch, i.e., ratio of propagate to total volume, to secure a high initial cell count. A growth media containing 1% yeast extract and 1% peptone is used as a nutrient and nitrogen source. Fermentations are monitored measuring xylose, xylulose, glucose and ethanol using HPLC-RI. Controls are included where xylose isomerase is added to the fermentation to determine the production of ethanol when xylose is not utilized.

1-35. (canceled)

36. A process of producing a fermentation product, which comprises the steps of:
a) pre-treating a lignocellulosic material to release or separate cellulose, hemicellulose and/or lignin,
b) subjecting the pre-treated material to a cellulase, and
c) fermenting in the presence of a fermenting organism,
wherein a xylose isomerase is added in step b) and/or step c).

37. A process of claim 36, which comprises the steps of:
a) pre-treating a lignocellulosic material to release or separate cellulose, hemicellulose and/or lignin,
b) subjecting the pre-treated material to a combination of cellulase and xylose isomerase, and
c) fermenting in the presence of a fermenting organism.

38. A process of claim 36, which comprises the steps of:
a) pre-treating a lignocellulosic material to release or separate cellulose, hemicellulose and/or lignin,
b) subjecting the pre-treated material to a cellulase, and
c) fermenting in the presence of a fermenting organism and xylose isomerase.

39. The process of claim 36, wherein the pre-treatment in step a) is carried out by subjecting lignocellosic material to chemical treatment and/or mechanical treatment.

40. The process of claim 39, wherein the chemical treatment in step a) is an acid treatment.

41. The process of claim 40, wherein the acid treatment in step a) is carried out using an organic acid.

42. The process of claim 39, wherein the pH is 1 to 5.

43. The process of claim 39, wherein the lignocellulosic material is acid treated with from 0.1 to 2.0 wt. % sulfuric acid.

44. The process of claim 39, wherein the mechanical treatment in step a) comprises treating the lignocellulosic material at a high temperature and/or a high pressure.

45. The process of claim 44, wherein the mechanical treatment in step a) is carried out under a pressure of 300-600 psi.

46. The process of claim 44, wherein the mechanical treatment in step a) is carried out at a temperature of 100-300° C.

47. The process of claim 36, wherein step a) is carried out as a dilute acid steam explosion, steam explosion, wet oxidation, or ammonia fiber explosion (or AFEX pretreatment).

48. The process of claim 36, wherein the released or separated cellulose, hemicellulose and/or lignin material obtained in step a) is treated with a hemicellulase to release xylose.

49. The process of claim 36, wherein step b) is carried in the presence of a hemicellulase.

50. The process of claim 36, wherein the cellulase is added in step b) to provide an activity level of 0.1-100 FPU per gram total solids (TS).

51. The process of claim 36, wherein the cellulase is added in step b) in an amount of 0.1-100 mg enzyme protein per gram total solids (TS).

52. The process of claim 36, wherein steps b) and c) are carried out simultaneously.

53. The process of claim 36, wherein the fermenting organism is yeast.

54. The process of claim 36, wherein the fermentation product is ethanol.

55. The process of claim 36, wherein steps b) and c) are carried out simultaneously.
